(19) United States
(12) Patent Application Publication
(10) Pub. No.: US 2007/0191941 A1
(43) Pub. Date: Aug. 16, 2007

(54) Capsular equatorial ring

(76) Inventors: Burkhard Dick, Mainz (DE); Olaf Morcher, Stuttgart (DE)

Correspondence Address: WILLIAM COLLARD;COLLARD & ROE, P.C., 1077 NORTHERN BOULEVARD, ROSLYN, NY, 11576, US

(21) Appl. No.: 10/583,757

(22) PCT Filed: May 11, 2005
(86) PCT No.: PCT/EP05/52131

§ 371 Date: Jun. 21, 2006

(30) Foreign Application Priority Data
Jun. 3, 2004 (DE) 102004027236.0

(51) Int. Cl. A61F 2/14

(52) U.S. Cl. 623/4.1; 623/6.16

(57) ABSTRACT

The invention relates to a capsular equatorial ring (5) which, after the removal of a natural lens, can be implanted in the opened capsular bag (3) of an eye and, when implanted, rests with its outer periphery against the inside of the capsular bag (3), essentially on the equator thereof, and radially stabilizes the capsular bag (3). The invention is characterized in that the capsular equatorial ring (5) is closed and has a number of foldable and/or creasable segments (7) and stiff segments (6) that are arranged alternately in the peripheral direction. The invention also relates to a capsular equatorial ring which is comprised, at least in part, of water-absorbable material and which is impregnated with an aqueous or water-soluble medicament.

The invention relates to a capsular equatorial ring which, after the removal of a natural lens, can be implanted in the opened capsular bag of an eye and, when implanted, rests with its outer periphery against the inside of the capsular bag, essentially on the equator thereof, and radially stabilizes the capsular bag.

Capsular equatorial rings are used for stabilizing the capsular bag in the eye. They are fitted as implants into the intact capsular bag and, for example after removal of the natural lens of an eye, are used to support the capsular tissue. After removal of the natural lens, for example on account of pronounced opacity, it is necessary that the opened capsular bag remains substantially in its original shape and in this way facilitates the implantation of an artificial intraocular lens. In cataract surgery, however, removal of the natural lens may result in damage to the zonular fiber tissue which secures the outside of the capsular bag in the region of its equator inside the eye. In order to avoid the associated deformations of the capsular bag or excessive stressing of the zonular fibers remaining undamaged, it is known to implant a capsular equatorial ring of the aforementioned type in the opened capsular bag. The capsular equatorial ring remains within the capsular bag during the operation and generally also after the insertion of an intraocular lens, and it presses against the tissue surrounding it in a ring shape.

Generally, the following indications may exist for implanting a capsular equatorial ring in the capsular bag:
local absence of zonular fibers, or damaged zonular fibers,
guarantee of consistent operating conditions,
luxation of an intraocular lens (IOL),
desired extension or spreading of the capsular bag,
stabilization of the capsular bag after removal of the lens in cases of high myopia,
zonulolysis,
pseudoexfoliation,
Marchesani syndrome, and
simplified implantation of foldable intraocular lenses.

Moreover, the implantation of the capsular equatorial ring affords the following advantages:
circular spreading of the capsular bag,
consistent operating conditions,
prevention of secondary cataract,
inhibition of capsular bag shrinkage,
minimizing or avoidance of capsular bag folds,
reduced clouding of the anterior capsule margin and thus better fundus visualization, e.g. in patients with problems affecting the retina.

EP 0 507 292 A1 discloses a closed capsular equatorial ring which, after the removal of a natural lens, can be implanted in the opened capsular bag of an eye. The capsular equatorial ring has a closed configuration and preferably has a rectangular or circular cross section. In a preferred embodiment, the inner periphery of the capsular equatorial ring is provided with a receiving groove into which a subsequently implanted intraocular lens engages and is thereby secured. The capsular equatorial ring is in principle formed in one piece.

DE 197 24 108 C1 discloses a capsular equatorial ring which is designed as a C-shaped, open, elastic spring clip with inwardly bent ends which, when the capsular equatorial ring is implanted, are brought close to one another counter to spring resistance in such a way that the capsular equatorial ring seeks to open out in the implanted state. Because of its spring clip characteristics, the capsular equatorial ring is thus able, on the one hand, to readily adapt to the size of the respective capsular bag and, on the other hand, is able to bear with notable tensioning on the equator of the capsular bag.

DE 199 51 148 A1 discloses a capsular tensioning ring with a curved elastic element, the ends of the elastic element being linked to one another and preferably overlapping. This ensures that the capsular tensioning ring, at least in the implanted and tensioned state, encloses an angle of more than 360° and thus acts counter to external tissue forces about a complete circle.

DE 202 06 342 U1 also discloses a capsular tensioning ring with an elastic element which has a closed shape in the implanted state. To generate the closed shape in the implanted state, each end of the elastic element has a head, of which a first head has a receiving seat pointing away from the element, and the second head has a tongue pointing away from the element and these are arranged in relation to one another in such a way that, when a force is exerted in the radial direction on the element, the tongue engages in the receiving seat and bears on the base of the receiving seat.

The present invention deals with the problem of providing an improved embodiment of a capsular equatorial ring of the type mentioned at the outset, in which in particular a reliable spreading of the capsular bag is guaranteed while at the same time ensuring minimal loading of the zonular tissue during and after implantation.

According to the invention, this problem is solved by the subject matter of the independent claim.

Advantageous embodiments are the subject matter of the dependent claims.

The invention is based on the general concept that a capsular equatorial ring which, after the removal of a natural lens, can be implanted in the opened capsular bag of an eye and, when implanted, rests with its outer periphery against the inside of the capsular bag, essentially on the equator thereof, and radially stabilizes the capsular bag, is to have a closed configuration and have a number of foldable and/or creasable segments and stiff segments. The foldable and/or creasable segments and the stiff segments are arranged alternately in the peripheral direction.

In this way, compared to a conventional, for example C-shaped capsular tensioning ring in which a guide eyelet results in considerable loading and stretching of the zonular fibers in the capsular bag equator, it is possible to achieve a capsular bag loading that is reduced and that can be individually adapted during the operation. Compared to the implantation of a conventional, rigid and open C-shaped capsular tensioning ring, the implantation of the capsular equatorial ring according to the invention is also much gentler on the tissue and therefore better tolerated. In conventional capsular tensioning rings, implantation may in rare cases lead to shearing of the zonular fibers in the implantation region and to pulling in an opposite zonular region. During implantation of the capsular tensioning ring, the zonular fibers are therefore subject to considerable loading both tangentially and also in the opposite region, which in the worst scenario leads to a dislocation of the stretched tensioning ring into the vitreous body. This makes explanation considerably more difficult. These disadvantages are not to be feared in the capsular equatorial ring according to the invention, because, during the implantation, this ring rests slowly and gently onto the capsular bag equator and, after the removal of the incision injector, it bears completely and independently on the capsular bag equator.

To implant the capsular equatorial ring, it can, for example, be folded once or folded twice and can even be implanted without any difficulty via a small-incision injector with an internal lumen diameter of only 1.4 mm. The double folding can provide even better adaptation of the outward deployment of the capsular equatorial ring and thus ensure a still gentler implantation. A feature of particular advantage is that it is not entirely necessary to use a viscoelastic for implanting the capsular equatorial ring according to the invention, although it can be used in order to avoid air bubbles.

In addition, the solution according to the invention affords the great advantage of being able to achieve a spreading of the capsular bag that is uniform, since it covers 360°, and the diameter of the foldable and/or creasable capsular equatorial ring is adapted optimally to an internal diameter of the opened capsular bag. This is of interest in particular in respect of rotation-stabilized or individually tailored intraocular lenses. Moreover, shrinkage of the capsular bag is greatly reduced compared to conventional open and rigid capsular tensioning rings.

The capsular equatorial ring expediently has a number of peripheral segments. It has proven particularly advantageous to use an arrangement of a total of 16 peripheral segments that are designed alternately as stiff PMMA segments (polymethyl methacrylate) and flexible hydrophilic HEMA/MMA copolymer segments (hydroxyethyl methacrylate-co-methyl methacrylate), hereinafter referred to as copolymer segments. The segmental design of the capsular equatorial ring ensures good foldability and creasability and, as a consequence, easier implantation of the capsular equatorial ring in the opened capsular bag.

PMMA is a material that has long been used in cataract surgery, and in particular in ophthalmic surgery, and is transparent, so that artificial lenses, for example, can also be made of this material, and, on the other hand, there have been years of experience in respect of the tolerability of this material. In addition, PMMA has a good inherent stiffness and a good shape memory which helps the implanted capsular equatorial ring to deploy independently and with precision in the capsular bag. By means of the good inherent stiffness, it is possible in particular to achieve better resistance to a shrinkage pressure of the capsular bag from outside.

By contrast, the copolymer segments ensure the good creasability and foldability of the capsular equatorial ring, readily permitting creasing of up to 180°. There have again been many years of experience with respect to the tolerability of the copolymer and of the polymers forming the copolymer, with the result that the capsular equatorial ring according to the invention is comprised only of materials for which there have been years of clinical testing and experience.

The capsular equatorial ring preferably has a sharp-edged outer periphery adjoining its end faces, in particular a sharp-edged anterior and posterior configuration. This double sharp-edged configuration permits in particular a 360° barrier for reduction of secondary cataract in the capsular bag periphery. In addition, the sharp-edged configuration results in a buckling and, consequently, a discontinuity at the capsular bag, preventing a proliferation of lenticular epithelial cells on the guiding or supporting structure formed by the capsular bag. With in vitro cultures of lenticular epithelial cells, it has been found that migration of these cells on one vessel wall is stopped at angular or sharp-edged buckled junctions to an adjoining vessel wall.

In a preferred embodiment of the solution according to the invention, an axial width of the outer periphery of the capsular equatorial ring is approximately 0.7 mm, the PMMA segments being approximately 0.5 mm wide in the segment center, and the copolymer segments being approximately 0.7 mm wide in the segment center. The axial width of the capsular equatorial ring at the. equator of the capsular bag is advantageous because, inter alia, the capsular equatorial ring is held in or forced into a parallel position with respect to the equatorial plane and thus leads to a uniform spreading of the capsular bag at the equatorial region, similar to when a natural lens is present. In addition, contact between anterior capsule membrane and posterior capsule membrane is counteracted, and the capsular bag is thus held open, and, at the same time, internal contact of the anterior capsule membrane with the posterior capsule membrane is prevented, as a result of which it is possible to achieve a reduction in capsular bag fibrosis.

The hydrophilic copolymer segments are expediently impregnated with a medicament. This in particular affords the advantage that, for example, a water-soluble medicament can be incorporated into the copolymer segments and, after implantation of the capsular equatorial ring, can be delivered slowly and uniformly. In addition, it is possible to achieve particularly exact dosing of the amount delivered and of the period of delivery, and this, compared to a conventional administration of medicament, is subject to much less fluctuation. At the same time, there is no need for a subsequent renewed intervention on the eye for administering medication.

Furthermore, the invention is based on the general concept that a capsular equatorial ring which, after the removal of a natural lens, can be implanted in the opened capsular bag of an eye, is to be comprised, at least in part, of water-absorbable material and to be impregnated with an aqueous or water-soluble medicament. In this way, it is possible to place a required medicament in the eye together with the capsular equatorial ring, and, in so doing, to achieve a particularly uniform and local distribution of the medicament. Since the materials used for the capsular equatorial ring according to the invention have already been clinically tested many times, there are many years of experience regarding their tolerability, such that a particularly well-tolerated implantation can be achieved. It is conceivable in this respect that the at least partially water-absorbable material, which is impregnated with the aqueous or water-soluble medicament, is arranged, for example, on an inner periphery or on an outer periphery of the capsular equatorial ring. Local or complete coating of the capsular equatorial ring with the water-absorbable material is also conceivable. The arrangement of the water-absorbable and medicament-impregnated material is possible both in the closed and foldable/creasable capsular equatorial ring according to the invention with segments and also in conventional, for example open, rigid and C-shaped capsular tensioning rings.

Further important features and advantages of the invention will become evident from the dependent claims, from the drawings, and from the associated description of the figures shown in the drawings.

It will be appreciated that the aforementioned features and the features still to be explained below can be used not only in the respectively cited combination, but also in other combinations or singly, without departing from the scope of the present invention.

A preferred illustrative embodiment of the invention is shown in the drawings and is explained in more detail in the following description.

In the drawings, which are schematic ones:

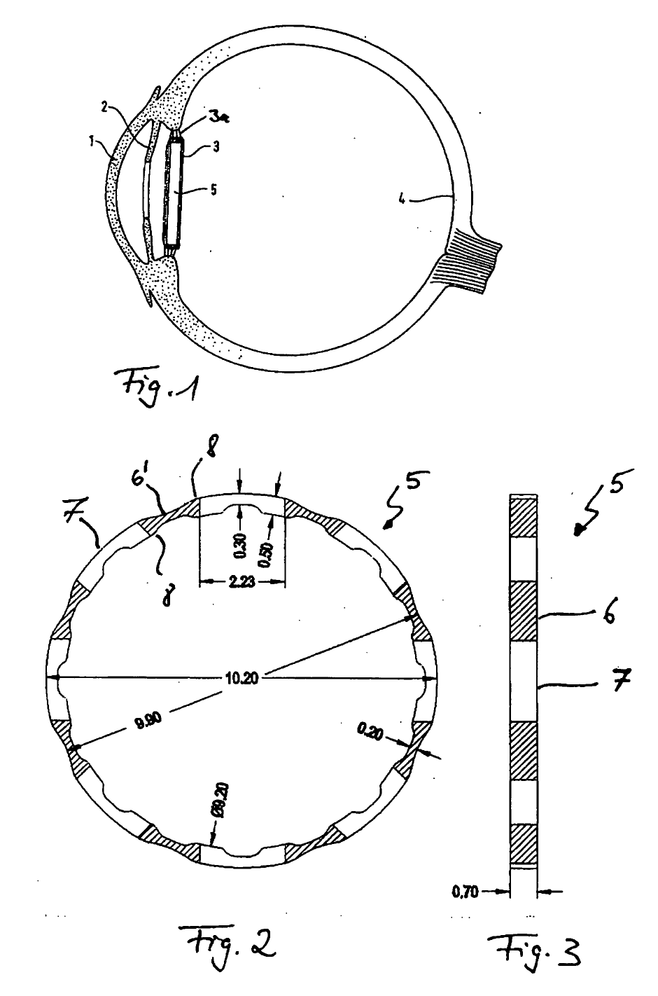

FIG. 1 shows a schematic longitudinal section through an eye, with a capsular equatorial ring implanted in the opened capsular bag,

FIG. 2 shows a capsular equatorial ring according to the invention in an axial elevation,

FIG. 3 shows a view corresponding to FIG. 2, but in a radial elevation.

According to FIG. 1, the eye shown there has, as is known, a cornea 1, an iris 2, a capsular bag 3 normally holding the natural lens, and a retina 4. In the example shown, the natural lens has been removed. For this purpose, the capsular bag 3 has been opened on its side directed toward the iris 2. In this operation, for example a cataract operation, zonular fibers 3a, which secure the capsular bag 3 at its equator inside the eye, may suffer a greater or lesser degree of damage. In order to avoid associated deformations of the capsular bag 3 and overloading of the undamaged zonular fibers 3a, a capsular equatorial ring 5 according to the invention can be inserted into the capsular bag 3. In particular, in cases where the zonule 3a is compromised, a capsular equatorial ring 5 of this kind is implanted in order to reduce further loading of the zonule.

According to FIGS. 2 and 3, the capsular equatorial ring is closed and has a number of foldable and/or creasable segments 7 and stiff segments 6 that are arranged alternately in the peripheral direction of the capsular equatorial ring 5. The capsular equatorial ring 5 is preferably made up of a total of 16 peripheral segments 6 and 7 designed alternately as stiff PMMA segments 6 (polymethyl methacrylate) and flexible hydrophilic HEMA/MMA copolymer segments 7 (hydroxyethyl methacrylate-co-methyl methacrylate). The stiff PMMA segments 6 are radially tapered toward the segment center at least from the inside, but preferably also from the outside, and they have a high inherent stiffness, which leads to an improved shape memory of the capsular equatorial ring 5 and additionally provides an increased resistance to the shrinkage pressure of the capsular bag from the outside. The stiff PMMA segments 6 are shown with hatching in FIGS. 2 and 3 and are at least approximately 0.2 mm thick and have an inwardly directed lateral transition zone 8.

The copolymer segments 7 likewise taper toward the segment center radially from the inside, resulting in the shape of a telephone receiver in the axial side view. The copolymer segments 7 have, for example, an approximately 28% water content. The HEMA interponates 7 are thicker compared to the PMMA segments 6 and slightly narrowed at the center in order to ensure the. foldability/creasability and the flexibility of the ring system.

The capsular equatorial ring 5 generally has a sharp-edged outer periphery adjoining its end faces, in particular a sharp-edged anterior or posterior configuration. The sharp-edged anterior or posterior configuration and the approximately 0.7 mm width of the PMMA segments 6 act against contact between anterior capsule membrane and posterior capsule membrane and thereby keep the capsular bag open, as a result of which it is possible to achieve a reduction in capsular bag fibrosis. The double sharp-edged configuration also permits a 360° barrier for secondary cataract reduction in the capsular bag periphery.

An axial width of the outer periphery of the capsular equatorial ring 5 is approximately 0.7 mm, the PMMA segments 6 being approximately 0.5 mm wide in the segment center, and the copolymer segments 7 being approximately 0.7 mm wide in the segment center.

Generally, the capsular equatorial rings 5 can be implanted using forceps or all available injector systems, so that no special implantation system is necessary. The capsular equatorial ring 5 can even be implanted without difficulty using a small-incision injector with an internal lumen diameter of only 1.4 mm, in which case the intraocular lens can also be implanted thereafter using the same implantation system.

The above-described injection system can be used to implant the capsular equatorial ring 5 folded once or folded twice, the double folding permitting better adaptation of the subsequent outward deployment of the capsular equatorial ring 5 in the capsular bag 3. A viscoelastic in the implantation system is not required for the implantation of the capsular equatorial ring 5, but it does avoid the occurrence of air bubbles during the implantation. If the capsular equatorial ring 5 is implanted more slowly, it opens out in an arc shape when making contact with the opposite capsular bag equator, as a result of which a particularly gentle implantation can be achieved.

Generally, the hydrophilic copolymer segments 7 are impregnated with a medicament and in this way have an additional function. In particular, this ensures that the administered medicament is released slowly and in a predefined dosage, without another form of administration of the medicament being required.

By implantation of the capsular equatorial ring 5 according to the invention, a predetermined capsular bag diameter can be achieved, which appears of interest with respect to rotation-stabilized or individually tailored intraocular lenses.

Generally, the capsular equatorial rings 5 can be produced with different diameters. The diameter can be 10.2 mm, for example.

Advantages of the capsular equatorial ring 5 according to the invention are in particular: the loading of the capsular bag can be reduced and individually adjusted during the operation; there is 360° secondary cataract reduction; the capsular bag is kept open all around because of the lateral height; reduced anterior capsule fibrosis for good fundus visualization of the retinal periphery; and uniform spreading of the capsular bag.

The fact that the PMMA and HEMA materials have already been used for a long time in ophthalmology means that it is thus possible to guarantee their good compatibility and it is possible to achieve a particularly gentle and patient-friendly implantation of the capsular equatorial rings 5.

Independently of the shape and structure of the capsular equatorial ring 5, the latter can be comprised, at least in part, of water-absorbable material, in which case it is impregnated with an aqueous or water-soluble medicament. In this case, it is possible in particular to imagine the capsular equatorial rings being coated with a water-absorbable material or being themselves made from this, or individual parts of them being made from this, as a result of which the capsular equatorial ring 5 can be used as a medicament carrier even when it has a different structure, for example if designed as a C-shaped tensioning ring.

Since the capsular equatorial ring lies radially outside the lens area required for vision, any possible discoloration of the ring by the medicament can be accepted.

In conclusion, the main features of the solution according to the invention can be characterized as follows:

The invention proposes that a capsular equatorial ring 5 which, after the removal of a natural lens, can be implanted in the opened capsular bag 3 of an eye, should be designed with a closed configuration and have a number of foldable and/or creasable segments 7 and stiff segments 6 that are arranged alternately in the peripheral direction of the capsular equatorial ring 5.

Compared to previous open and rigid tensioning rings, the implantation of the capsular equatorial ring 5 according to the invention leads to a reduced and individually adjustable loading of the capsular bag and to reduced anterior capsule fibrosis and uniform spreading of the capsular bag.

1. A capsular equatorial ring (5) which, after the removal of a natural lens, can be implanted in the opened capsular bag (3) of an eye and, when implanted, rests with its outer periphery against the inside of the capsular bag (3), essentially on the equator thereof, and radially stabilizes the capsular bag (3), wherein the capsular equatorial ring (5) is closed and has a number of foldable and/or creasable segments (7) and stiff segments (6) that are arranged alternately in the peripheral direction.

2. The capsular equatorial ring as claimed in claim 1, wherein the capsular equatorial ring (5) has 16 peripheral segments (6, 7).

3. The capsular equatorial ring as claimed in claim 1, wherein the peripheral segments (6, 7) are designed alternately as stiff PMMA segments (6) (polymethyl methacrylate) and HEMA/MMA copolymer segments (7) (hydroxyethyl methacrylate-co-methyl methacrylate).

4. The capsular equatorial ring as claimed in claim 3, wherein the PMMA segments (6) taper radially toward the segment center at least from the inside.

5. The capsular equatorial ring as claimed in claim 3, wherein the PMMA segments (6) taper in the axial direction of the ring (5) toward the segment center.

6. The capsular equatorial ring as claimed in claim 3, wherein the HEMA/MMA copolymer segments (7) taper radially toward the segment center from the inside.

7. The capsular equatorial ring as claimed in claim 3, wherein the HEMA/MMA copolymer segments (7) have an approximately 28% water content.

8. The capsular equatorial ring as claimed in claim 3, wherein a radial thickness of the PMMA segments (6) in the segment center is approximately 0.2 mm.

9. The capsular equatorial ring as claimed in claim 3, wherein an axial width of the outer periphery of the capsular equatorial ring (5) is approximately 0.7 mm, the PMMA segments (6) being approximately 0.5 mm wide in the segment center, and the HEMA/MMA copolymer segments (7) being approximately 0.7 mm wide in the segment center.

10. The capsular equatorial ring as claimed in claim 1, wherein the capsular equatorial ring (5) has a sharp-edged outer periphery adjoining its end faces, in particular a sharp-edged anterior and posterior configuration.

11. The capsular equatorial ring as claimed in claim 3, wherein the HEMA/MMA copolymer segments (7) are impregnated with a medicament.

12. (canceled)
